(19) United States
(12) Patent Application Publication
(10) Pub. No.: US 2025/0186802 A1
(43) Pub. Date: Jun. 12, 2025

(54) Y-ZONE TREATMENT DEVICE USING PULSE-CONTROLLED LED

(71) Applicant: MEDMIX CO., LTD., Incheon (KR)

(72) Inventors: Soo Jeong LIM, Chuncheon-si (KR); Yun Seok YU, Incheon (KR)

(21) Appl. No.: 18/847,203

(22) PCT Filed: Mar. 11, 2023
(86) PCT No.: PCT/KR2023/003339

§ 371 Date: Sep. 14, 2024

(30) Foreign Application Priority Data
Mar. 16, 2022 (KR) 10-2022-0032581

(51) Int. Cl. A61N 5/06

(52) U.S. Cl. CPC A61N 5/0625; A61N 2005/0626; A61N 2005/0645; A61N 2005/0651; A61N 2005/0659; A61N 2005/0663

(57) ABSTRACT

A Y-zone treatment device using a pulse-controlled LED, presented in the present invention, comprises: a Y-zone treatment unit, which generates an LED light for female Y-zone treatment at the position of a female Y-zone area; and a driving control unit for controlling the driving of the Y-zone treatment unit, which generates the LED light for female Y-zone treatment at the position of the female Y-zone area, and controlling pulse width by pulse width modulation (PWM) for driving of the Y-zone treatment unit, and thus the female Y-zone can be treated with light emission of an LED in the female Y-zone area by wearing the device on the female Y-zone area, and the temperature can be controlled by means of pulse width control of PWM.

### TECHNICAL FIELD

The present disclosure relates to a Y-zone treatment device using a pulse-controlled light emitting diode (LED) and, more particularly, to a Y-zone treatment device using a pulse-controlled LED which is capable of treating a female Y-zone by light emission of an LED to the female Y-zone area by wearing the device on the female Y-zone area, and controls a temperature to enable a heating function and low-temperature burn prevention.

### BACKGROUND ART

As the trend of wearing leggings has increased recently, many care products for a female Y-zone are being released. These female Y-zone care products include female vaginal lactobacillus and Y-zone care cosmetic products.

Y-zone care, which is a concern for women, is caused by tight pants and long sitting times, which leads to irritation in the Y-zone, secretions due to the irritation, and anxiety, so many women are taking care of their Y-zones. That is, when the Y-zone is not properly managed, it may cause vaginitis, itchiness, and unpleasant odor, so many women are paying more attention to hygiene and taking care of their Y-zones.

The existing Y-zone care of a woman generally uses a cleanser to clean and moisturize the Y-zone, and is often used in the form of lotions or sprays. However, these Y-zone care products, which are simply cleansing products for cleaning or moisturizing, have limitations in long-term Y-zone care, and in particular, have a problem that there are time and space constraints on the use of Y-zone care.

### DISCLOSURE

### Technical Problem

The present disclosure has been proposed to solve the above problems of existing proposed methods and is intended to propose a Y-zone treatment device using a pulse-controlled LED which includes a Y-zone treatment unit which generates LED light for female Y-zone treatment at a position of a female Y-zone area, and a driving control unit which controls driving of the Y-zone treatment unit, which generates the LED light for the female Y-zone treatment at the position of the female Y-zone area, and controls a pulse width in pulse width modulation (PWM) for the control of the driving of the Y-zone treatment unit, thereby enabling the female Y-zone treatment by light emission of an LED to the female Y-zone area by wearing the device on the female Y-zone area, and enabling temperature control by pulse width control of PWM.

In addition, the present disclosure is intended to propose a Y-zone treatment device using pulse-controlled LED, which controls the driving of an LED light source unit of the Y-zone treatment unit so that a temperature thereof does not rise to a specific temperature or more by the pulse width control of the PWM of the driving control unit, thereby enabling a heating function and low-temperature burn prevention when the device is used on the Y-zone skin of a woman.

In addition, the present disclosure is intended to propose a Y-zone treatment device using a pulse-controlled LED which enables a disposable pad to be used by being attached to the Y-zone treatment unit having the LED light source unit controlled so that a temperature thereof does not rise to a specific temperature or more by the pulse width control of PWM of the driving control unit, thereby further improving cleanliness through periodic replacement use in the sensitive Y-zone area of a woman and personal hygiene according thereto.

### Technical Solution

In order to accomplish the above objectives, a Y-zone treatment device using a pulse-controlled LED according to features of the present disclosure,
as the Y-zone treatment device using a pulse-controlled LED,
includes: a Y-zone treatment unit that generates LED light for female Y-zone treatment at a position of a female Y-zone area; and
a driving control unit that controls driving of the Y-zone treatment unit, which generates the LED light for the female Y-zone treatment at the position of the female Y-zone area, and controls a pulse width in pulse width modulation (PWM) for the control of the driving of the Y-zone treatment unit.

Preferably, the Y-zone treatment unit
may include: a Y-zone treatment unit body that is capable of being worn inside female underwear and being used at the position of the female Y-zone area; and
an LED light source unit that is disposed at a center portion of the Y-zone treatment unit body and generates the LED light for the female Y-zone treatment.

More preferably, the Y-zone treatment unit body (111)
may be made of medical silicone that is harmless to a human body.

More preferably, the Y-zone treatment unit body
may be made of the medical silicone that is harmless to the human body and may be configured in a flexible form to be worn in the female underwear and located in the female Y-zone area.

Preferably, the LED light source unit
may emit light with the LED for the female Y-zone treatment at the position of the female Y-zone area, and may be controlled so that a temperature thereof does not rise to a specific temperature or more through pulse width control which is control in pulse width modulation (PWM) by the driving control unit

More preferably, the specific temperature
may be in a temperature range of 37 to 39 degrees in which a low-temperature burn is prevented and a heating function is provided at the position of the female Y-zone area by the LED light emission of the LED light source unit.

More preferably, the LED light source unit
may be arranged in the center portion of the Y-zone treatment unit body and may be configured as a single wavelength LED light source that generates the LED light for the female Y-zone treatment.

More preferably, the LED light source unit
may be arranged in the center portion of the Y-zone treatment unit body and include a BLUE LED, a RED LED, and an IR LED that generate three LED wavelengths for the female Y-zone treatment.

### Advantageous Effects

The Y-zone treatment device using a pulse-controlled LED proposed in the present disclosure includes the Y-zone treatment unit which generates LED light for female Y-zone treatment at the position of the female Y-zone area, and the driving control unit which controls the driving of the Y-zone treatment unit, which generates the LED light for the female Y-zone treatment at the position of the female Y-zone area, and controls a pulse width in pulse width modulation (PWM) for the control of the driving of the Y-zone treatment unit, thereby enabling the female Y-zone treatment by light emission of an LED to the female Y-zone area by wearing the device on the female Y-zone area, and enabling temperature control by pulse width control of PWM.

In addition, according to the Y-zone treatment device using a pulse-controlled LED of the present disclosure, the driving of an LED light source unit of the Y-zone treatment unit is controlled so that a temperature thereof does not rise to a specific temperature or more by the pulse width control of the PWM of the driving control unit, thereby enabling a heating function and low-temperature burn prevention when the device is used on the Y-zone skin of a woman.

In addition, according to the Y-zone treatment device using a pulse-controlled LED of the present disclosure, disposable pads are enabled to be used by being attached to the Y-zone treatment unit having the LED light source unit controlled so that a temperature thereof does not rise to a specific temperature or more by the pulse width control method of the PWM of the driving control unit, thereby further improving cleanliness through periodic replacement use in the sensitive Y-zone area of a woman and personal hygiene according thereto.

### DESCRIPTION OF DRAWINGS

FIG. 1 is a diagram illustrating the configuration of a Y-zone treatment device using a pulse-controlled LED according to an embodiment of the present disclosure as a functional block.

FIG. 2 is a diagram illustrating the configuration of a Y-

zone treatment unit of the Y-zone treatment device using a pulse-controlled LED according to an embodiment of the present disclosure as a functional block.

FIG. 3 is a diagram illustrating the configuration of an LED light source unit of the Y-zone treatment device using a pulse-controlled LED according to an embodiment of the present disclosure as a functional block.

FIG. 4 is a diagram illustrating the Y-zone skin treatment and control method of the Y-zone treatment device using a pulse-controlled LED according to an embodiment of the present disclosure.

FIG. 5 is a view illustrating the schematic configuration of the Y-zone treatment device using a pulse-controlled LED according to an embodiment of the present disclosure.

### DESCRIPTION OF THE REFERENCE NUMERALS IN THE DRAWINGS

100: Y-zone treatment device according to an embodiment of the present disclosure
101: Pad
110: Y-zone treatment unit
111: Y-zone treatment unit body
112: LED light source unit
112a: BLUE LED
112b: RED LED
112c: IR LED
120: Driving control unit

### BEST MODE

Hereinafter, preferred embodiments are described in detail with reference to the attached drawings so that those skilled in the art to which the present disclosure belongs can easily practice the present disclosure. However, when describing the preferred embodiments of the present disclosure in detail, if it is determined that a specific description of a related known function or configuration may unnecessarily obscure the gist of the present disclosure, the detailed description is omitted. In addition, parts that perform similar functions and actions are denoted by the same symbols throughout the drawings.

Additionally, throughout the specification, when a part is said to be ‘connected’ to another part, this includes not only a case in which they are ‘directly connected’, but also a case in which they are ‘indirectly connected’ with still another element placed therebetween. In addition, to ‘include’ an element means, unless otherwise specifically stated, that it may include other elements, but not to the exclusion of other elements.

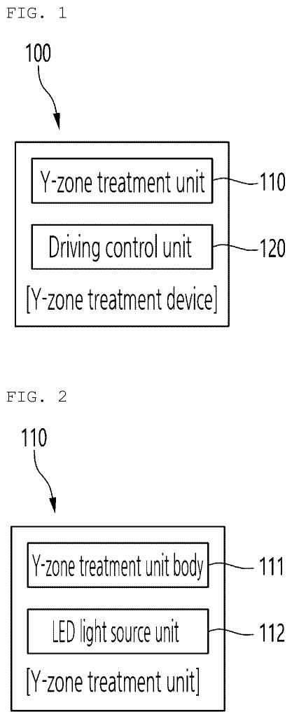

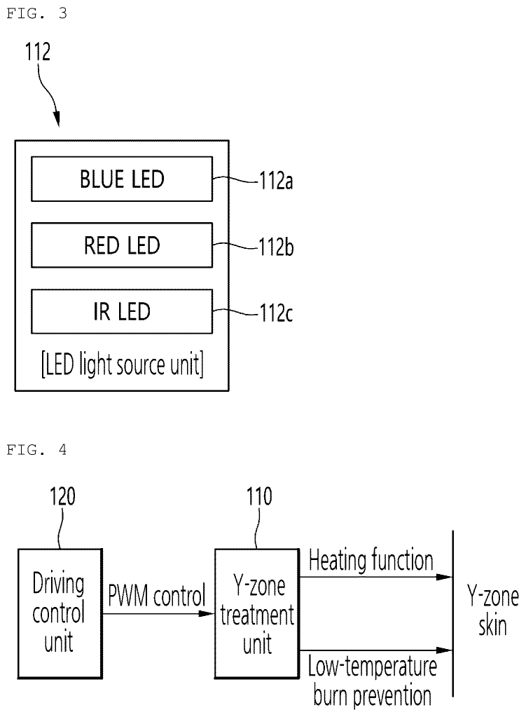

FIG. 1 is a diagram illustrating the configuration of a Y-zone treatment device using a pulse-controlled LED according to an embodiment of the present disclosure as a functional block, FIG. 2 is a diagram illustrating the configuration of a Y-zone treatment unit of the Y-zone treatment device using a pulse-controlled LED according to an embodiment of the present disclosure as a functional block, FIG. 3 is a diagram illustrating the configuration of an LED light source unit of the Y-zone treatment device using a pulse-controlled LED according to an embodiment of the present disclosure as a functional block, FIG. 4 is a diagram illustrating the Y-zone skin treatment and control method of the Y-zone treatment device using a pulse-controlled LED according to an embodiment of the present disclosure, view illustrating the schematic configuration of the Y-zone treatment device using a pulse-controlled LED according to an embodiment of the present disclosure. As illustrated in each of FIGS. 1 to 5, a Y-zone treatment device 100 using a pulse-controlled LED according to an embodiment of the present disclosure may include a Y-zone treatment unit 110 which generates LED light for the female Y-zone treatment at the position of a female Y-zone area, and a driving control unit 120 which controls the driving of the Y-zone treatment unit 110, which generates the LED light for the female Y-zone treatment at the position of the female Y-zone area, and controls a pulse width in pulse width modulation (PWM) for the control of the driving of the Y-zone treatment unit 110. Hereinafter, the specific configuration of the Y-zone treatment device using a pulse-controlled LED according to an embodiment of the present disclosure will be described in detail with reference to the accompanying drawings.

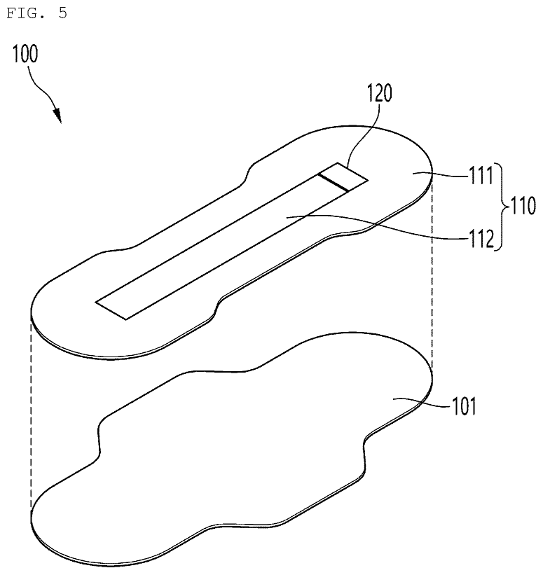

The Y-zone treatment unit 110 is configured to generate LED light for the female Y-zone treatment at the position of the female Y-zone area. As illustrated in each of FIGS. 2 and 5, the Y-zone treatment unit 110 include a Y-zone treatment unit body 111 that may be worn inside female underwear and be used at the position of the female Y-zone area, and an LED light source unit 112 that is disposed at the center portion of the Y-zone treatment unit body 111 and generates LED light for the female Y-zone treatment. Here, as illustrated in FIG. 5, the Y-zone treatment unit 110 may be used by being attached to a pad 101, wherein the pad 101 is a disposable consumable that may be replaced to cover the Y-zone treatment unit 110, which generates LED light for the female Y-zone treatment at the position of the female Y-zone area.

In addition, the Y-zone treatment unit body 111 may be made of medical silicone that is harmless to the human body. The Y-zone treatment unit body 111, which is made of medical silicone harmless to the human body, may be formed in a flexible form to be worn inside female underwear and be positioned in the female Y-zone area. Here, the Y-zone treatment unit body 111 may be configured as a rectangular structure in the shape of a sanitary pad that may be worn inside the female underwear and positioned in the female Y-zone area, and may be configured as a structure in which rounded grooves are formed in the center portions of opposite sides in a longitudinal direction of the Y-zone treatment unit body.

In addition, the LED light source unit 112 may emit light with an LED for the female Y-zone treatment at the position of the female Y-zone area and may be controlled so that a temperature thereof does not rise to a specific temperature or more through pulse width control which is control in pulse width modulation (PWM) by the driving control unit 120. Here, the specific temperature may be in a temperature range of 37 to 39 degrees in which a low-temperature burn is prevented and a heating function is provided at the position of the female Y-zone area by LED light emission of the LED light source unit 112.

In addition, the LED light source unit 112 may be arranged in the center portion of the Y-zone treatment unit body 111 and may be configured as a single wavelength LED light source that generates LED light for the female Y-zone treatment.

In addition, the LED light source unit 112 may be arranged in the center portion of the Y-zone treatment unit body 111, and may include a BLUE LED 112a, a RED LED 112b, and an IR LED 112c that generate three LED wavelengths for the female Y-zone treatment.

The driving control unit 120 may control the driving of the Y-zone treatment unit 110, which generates LED light for female Y-zone treatment at the position of the female Y-zone area and may control a pulse width in pulse width modulation (PWM) for the control of the driving of the Y-zone treatment unit 110. The driving control unit 120 may control the driving of the LED light source unit 112 of the Y-zone treatment unit 110, which generates LED light for the female Y-zone treatment at the position of the female Y-zone area, so that a temperature thereof does not rise to a specific temperature of 37 to 39 degrees or more by the pulse width control of pulse width modulation (PWM). Here, the driving control unit 120 uses a frequency of 60 Hz to 1000 Hz and controls the driving of the LED light source unit 112 so that a temperature thereof does not rise to a specific temperature or more, thereby preventing a low-temperature burn and providing a heating function at the position of the female Y-zone area.

As described above, the Y-zone treatment device using a pulse-controlled LED according to an embodiment of the present disclosure includes the Y-zone treatment unit which generates LED light for female Y-zone treatment at the position of the female Y-zone area, and the driving control unit which controls the driving of the Y-zone treatment unit, which generates the LED light for the female Y-zone treatment at the position of the female Y-zone area, and controls a pulse width in pulse width modulation (PWM) for the control of the driving of the Y-zone treatment unit, thereby enabling the female Y-zone treatment by through light emission of an LED to the female Y-zone area by wearing the device on the female Y-zone area, and enabling temperature control by pulse width control of PWM. Particularly, the driving of the LED light source unit of the Y-zone treatment unit is controlled so that a temperature thereof does not rise to a specific temperature or more by the pulse width control of PWM of the driving control unit, thereby enabling a heating function in and low-temperature burn prevention when the device is used on the Y-zone skin of a woman. In addition, a disposable pad is allowed to be used by being attached to the Y-zone treatment unit having the LED light source unit controlled so that a temperature thereof does not rise to a specific temperature or more by the pulse width control of PWM of the driving control unit, thereby further improving cleanliness through periodic replacement use in the sensitive Y-zone area of a woman and personal hygiene according thereto.

The present disclosure described above may be modified or applied in various ways by those skilled in the art to which the present disclosure belongs, and the scope of the technical idea according to the present disclosure should be determined by the following patent claims.

1. A Y-zone treatment device (100) using a pulse-controlled LED, the device comprising:
a Y-zone treatment unit (110) that generates LED light for female Y-zone treatment at a position of a female Y-zone area; and
a driving control unit (120) that controls driving of the Y-zone treatment unit (110), which generates the LED light for the female Y-zone treatment at the position of the female Y-zone area, and controls a pulse width in pulse width modulation (PWM) for the control of the driving of the Y-zone treatment unit (110).

2. The device of claim 1, wherein the Y-zone treatment unit (110) comprises:
a Y-zone treatment unit body (111) that is capable of being worn inside female underwear and being used at the position of the female Y-zone area; and
an LED light source unit (112) that is disposed at a center portion of the Y-zone treatment unit body (111) and generates the LED light for the female Y-zone treatment.

3. The device of claim 2, wherein the Y-zone treatment unit body (111) is made of medical silicone that is harmless to a human body.

4. The device of claim 3, wherein the Y-zone treatment unit body (111) is made of the medical silicone that is harmless to the human body and is configured in a flexible form to be worn in the female underwear and located in the female Y-zone area.

5. The device of claim 2, wherein the LED light source unit (112) emits light with the LED for the female Y-zone treatment at the position of the female Y-zone area, and is controlled so that a temperature thereof does not rise to a specific temperature or more through pulse width control which is control in pulse width modulation (PWM) by the driving control unit (120).

6. The device of claim 5, wherein the specific temperature is in a temperature range of 37 to 39 degrees in which a low-temperature burn is prevented and a heating function is provided at the position of the female Y-zone area by the LED light emission of the LED light source unit (112).

7. The device of claim 6, wherein the LED light source unit (112) is arranged in the center portion of the Y-zone treatment unit body (111) and is configured as a single wavelength LED light source that generates the LED light for the female Y-zone treatment.

8. The device of claim 6, wherein the LED light source unit (112) is arranged in the center portion of the Y-zone treatment unit body (111) and comprises a BLUE LED (112a), a RED LED (112b), and an IR LED (112c) that generate three LED wavelengths for the female Y-zone treatment.
